(19) United States
(12) Patent Application Publication
(10) Pub. No.: US 2017/0269066 A1
(43) Pub. Date: Sep. 21, 2017

(54) SKIN ORGAN CULTURE MODEL SYSTEM AND ITS USE

(71) Applicant: Galderma Research & Development, Biot (FR)

(72) Inventor: Hanan OSMAN-PONCHET, Antibes (FR)

(21) Appl. No.: 15/329,807

(22) PCT Filed: Jul. 28, 2015
(86) PCT No.: PCT/EP2015/067252

§ 371 Date: Jan. 27, 2017

Related U.S. Application Data
(60) Provisional application No. 62/030,649, filed on Jul. 30, 2014.

(51) Int. Cl. G01N 33/50

(52) U.S. Cl. CPC G01N 33/5088; G01N 33/5044; G01N 33/5023

(57) ABSTRACT

A skin organ culture model system is provided having a perfusion plate (1), a peristaltic pump (2), and a culture reservoir (3). The skin organ culture model system combines both the dynamic perfusion of culture medium with the use of plasma as culture medium in order to provide, in the model system, culture conditions that are similar to in vivo conditions.

### FIELD OF THE INVENTION

The present invention relates to a skin organ culture model system and its use to evaluate skin penetration (absorption and distribution) of xenobiotics, topical drug metabolism, drug-drug interactions, drug-sunscreen interactions, inflammatory skin reaction, the efficacy of sunscreen or effect of UV exposure, the effect of various photo-protective drugs intended to prevent or treat skin cancer, the immunological function of skin cells by measuring cytokine secretion in the culture medium, the efficacy and safety of cosmetic ingredients and drugs, or the efficacy of cosmetic “anti-aging” skin care by measuring collagen synthesis.

### BACKGROUND OF THE INVENTION

Human skin organ culture models have been extensively used in the past since their introduction early last century, as these culture models allow maintaining the architecture and the tissue organization.

Providing an environment sufficient for the development of skin organ cultures can be used to study biological processes, dermal toxicity, and penetration and metabolism of xenobiotics in intact skin [1]. Furthermore, within certain limits, processes responsible for repair and regeneration of damaged skin can also be studied in the model disclosed in reference [1] because the skin can be cultured for several days.

Despite their potential advantages, known skin organ culture models have some technical limitations leading to a poor in vitro—in vivo correlation, thus limiting their use in biomedical research.

Two different protocols of skin organ culture can be used [2]. In the first protocol, skin punch biopsies are cultured in wells of a culture plate in a synthetic or semisynthetic culture medium (immersed skin organ culture model), as shown in FIG. 1. In the second protocol, skin discs are cultured on a microporous membrane of an insert, allowing transport of culture medium via the dermis into the epidermis, with the epidermal side remaining free of direct contact with the culture medium (two-compartment skin organ culture model) as shown in FIG. 2. In both protocols, fresh culture medium is replaced at one day or two day intervals (static conditions).

However, under natural conditions, tissues and organs of the body are continuously perfused by the blood circulatory and lymphatic systems, which together ensure a constant refreshment of nutrients and removal of waste products. In skin organ culture models, dynamic perfusion of culture medium seems to be essential for nutrient and oxygen exchange across skin tissues. In addition, the composition of the synthetic culture media used in skin organ cultures does not correspond exactly to human blood or plasma composition.

Recently, organ culture models have been developed with dynamic perfusion of culture medium, such as the dynamic chip-based bioreactor using skin equivalent culture [3]. However, to the best of our knowledge, there is no skin organ culture model that combines both the dynamic perfusion of culture medium with the use of human plasma as culture medium to provide conditions similar to in vivo conditions.

### DESCRIPTION OF THE INVENTION

The invention provides a novel skin organ culture model system that combines both the dynamic perfusion of culture medium with the use of plasma, preferably human plasma, as culture medium, in order to provide culture conditions similar to in vivo conditions.

In one general aspect, the invention relates to a skin organ culture model system comprising:
(i) a perfusion plate (1) comprising at least one skin biopsy;
(ii) a peristaltic pump (2); and
(iii) a culture medium reservoir (3) comprising a culture medium, and connected to the perfusion plate (1) and the peristaltic pump (2),
   wherein the culture medium is plasma, and the peristaltic pump (2) pumps the culture medium from the culture reservoir (3) into and out of the perfusion plate at a flow rate ranging from 0.1 mL/min to 20 mL/min, and preferably from 9 mL/min to 13 mL/min.

In a particular embodiment of the invention, a skin organ culture model system is as shown in FIG. 6. In another particular embodiment, the perfusion plate (1) can be a plate as shown in FIG. 4.

In other general aspects, the invention relates to use of a skin organ culture model system according to the invention to evaluate skin penetration (absorption and distribution) of xenobiotics; topical drug metabolism; drug-drug interactions; drug-sunscreen interactions; inflammatory skin reaction; the efficacy of sunscreen or effect of UV exposure; the effect of various photo-protective drugs intended to prevent or treat skin cancer; the immunological function of skin cells by measuring cytokine secretion in the culture medium; the efficacy and safety of cosmetic ingredients and drugs; or the efficacy of cosmetic “anti-aging” skin care by measuring collagen synthesis; and use of a skin organ culture model system according to the invention to identify biomarkers responsible for repair and regeneration of damaged skin.

In yet other general aspects, the invention relates to methods of evaluating skin penetration (absorption and distribution) of xenobiotics; topical drug metabolism; drug-drug interactions; drug-sunscreen interactions; inflammatory skin reaction; the efficacy of sunscreen or effect of UV exposure; the effect of various photo-protective drugs intended to prevent or treat skin cancer; the immunological function of skin cells by measuring cytokine secretion in the culture medium; the efficacy and safety of cosmetic ingredients and drugs; or the efficacy of cosmetic “anti-aging” skin care by measuring collagen synthesis; and use of a skin organ culture model system according to the invention to identify biomarkers responsible for repair and regeneration of damaged skin, and methods of identifying biomarkers responsible for repair and regeneration of damaged skin comprising maintaining a skin organ culture model system according to the invention in a cell incubator set at 37° C. and 5% CO2 for a duration of 3 to 7 days.

Methods according to embodiments of the invention can further comprise measuring a level of gene expression of one or more genes selected from the group consisting of drug transporter genes and drug metabolism genes.

This invention also aims to develop new technically easier conditions which are closer to, or more similar to in vivo conditions, in order to provide an enhanced human skin organ culture model system leading to better in vitro—in vivo correlation and better prediction of clinical responses.

Namely, the present invention developed a skin organ culture method using dynamically perfused human plasma to replace synthetic culture medium used in static conditions, in order to better mimic physiological conditions. The new conditions applied to human skin organ culture increase the expression of drug transporter and drug metabolizing enzyme genes. This quick and simple system can be used in a cell incubator set at 37° C. and 5% CO₂ for a duration of 3 to 7 days to evaluate skin penetration (absorption and distribution) of xenobiotics; topical drug metabolism; drug-drug interactions; drug-sunscreen interactions; inflammatory skin reaction; the efficacy of sunscreen or effect of UV exposure; or effect of various photo-protective drugs intended to prevent or treat skin cancer. It can also be used to evaluate the immunological function of skin cells by measuring cytokine secretion in the culture medium, or the efficacy and safety of cosmetic ingredients and drugs or the efficacy of cosmetic “anti-aging” skin care by measuring collagen synthesis.

This quick and simple system can also be used to study pharmacology or dermal toxicity, or penetration and metabolism of xenobiotic in skin biopsies maintained in culture conditions very close to their natural microenvironment.

Some tests have been realized to develop and validate this model. The invention is further described by the Figures and following Examples, none of which shall be construed as limiting the scope of the appended claims.

### BRIEF DESCRIPTION OF THE SEVERAL VIEWS OF THE DRAWINGS

In the drawings:

FIG. 1 shows a photographic image of an immersed skin organ culture model;

FIG. 2 shows a photographic image of a two-compartment skin organ culture model. A: Insert unit with a microporous membrane; B: Receiver compartment containing culture medium; C: Skin; D: Donor compartment

FIG. 3 shows the results of the experiment described in Example 1 to determine the effect of plasma on gene expression in a skin organ culture model; gene expression levels of SLC47A1 and CYP1A1 were determined by real time PCR; in the experiment, human skin punch biopsies each 6 mm in diameter were cultured in the wells of a 6-well culture plate in either a synthetic culture medium (Skin long term culture medium, Biopredic, France) or in human plasma for 3 days;

FIG. 4 is a photographic image of a Reinnervate perfusion plate with four skin biopsies per well;

FIG. 5 is a schematic representation of unidirectional dynamic flow of culture medium in a perfusion plate;

FIG. 6 is a photograph of a skin organ culture system according to an embodiment of the invention; skin organ culture is in a perfusion plate; the system includes a perfusion plate (1), a peristaltic pump (2), and a culture medium reservoir (3);

FIG. 7 shows the results of the experiment described in Example 2 to determine the effect of culture medium perfusion on gene expression in skin organ culture model; human skin samples were cultured under dynamic culture medium perfusion conditions at different flow rates for a duration of 3 days in skin long term culture medium; gene expression of drug metabolism and ABC and SLC transporters was analyzed by real time PCR; each flow rate was tested in triplicate in one separate experiment using a different skin donor; results concerning the flow rate 0 mL/min represent the mean and SEM of three different experiments performed on three different donors; results of the others flow rates represent the mean and SEM of triplicates on one single donor;

FIG. 8 shows the results of the experiment described in Example 3 to determine the effect of substitution of culture medium with human plasma on SLC47A1 gene expression in skin organ culture model; human skin samples were cultured for 3 days in skin long term culture medium or in human plasma under static or dynamic perfusion conditions (flow rate 11 mL/min); gene expression of SLC47A1, a member of SLC transporter family, was analyzed by real time PCR; the experiment was done in triplicate with one single donor identified as P15; FIG. 8A shows results from dynamic culture conditions; FIG. 8B shows results from static culture conditions;

FIG. 9 shows the results of an experiment to determine the effect of substitution of static conditions with dynamic perfusion conditions of culture medium on SLC47A1 gene expression in skin organ culture model; human skin samples were cultured either in dynamic culture medium perfusion at a flow rate of 11 mL/min, or in static conditions for 3 days in skin long term culture medium or in human plasma; gene expression of SLC47A1, a member of SLC transporter family was analyzed by real time PCR; the experiment was done in triplicate with one single donor identified as P15; FIG. 9A shows results from human plasma; FIG. 9B shows results from skin long term culture medium; and

FIG. 10 shows the results of an experiment to determine the effect of a combination of both culture medium perfusion and the use of human plasma as culture medium on SLC47A1 gene expression in skin organ culture model; human skin samples were cultured in dynamic culture medium perfusion at a flow rate of 11 mL/min or in static conditions for 3 days in skin long term culture medium or in human plasma; gene expression of SLC47A1, a member of SLC transporter family, was analyzed by real time PCR; and the experiment was done in triplicate with one single donor identified a P15.

### EXAMPLES

### Example 1

### Effect of Plasma on Gene Expression in Skin Organ Culture Model.

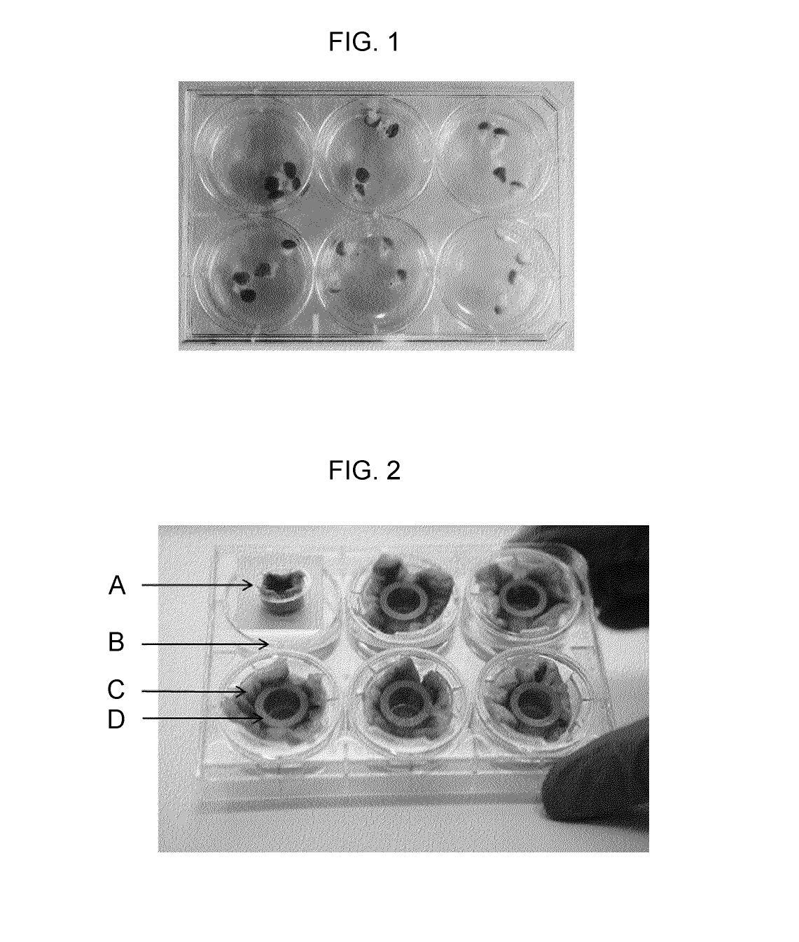

Skin punch biopsies each 6 mm in diameter were cultured in the wells of a 6-well culture plate in a synthetic culture medium (Skin long term culture medium, Biopredic, France). In this immersed organ culture model, 4 skin biopsies were cultured per well (see FIG. 1). Each well was filled with 5 mL of synthetic culture medium. The 6-well plate was maintained in a cell incubator set at 37° C., 5% CO₂, and high humidity. Fresh synthetic culture medium was provided every day for a duration of 3 days.

In parallel, skin punch biopsies each 6 mm in diameter obtained from the same donor were cultured under the same conditions, except that the culture medium was replaced by a human plasma sample. The human plasma was changed every day for a duration of 3 days.

At the end of the 3-day culture period, skin biopsies were washed using sterile PBS buffer, and total RNA was extracted from the skin samples. Gene expression of the SLC47A1 gene coding for multidrug and toxin extrusion protein 1 (MATE1 transporter), and the CYP1A1 gene, which codes for a member of the cytochrome P450 superfamily of enzymes, was analyzed by real time PCR as described in Example 7 below.

Three different experiments were performed using skin samples from three different donors. Each condition was tested in triplicate.

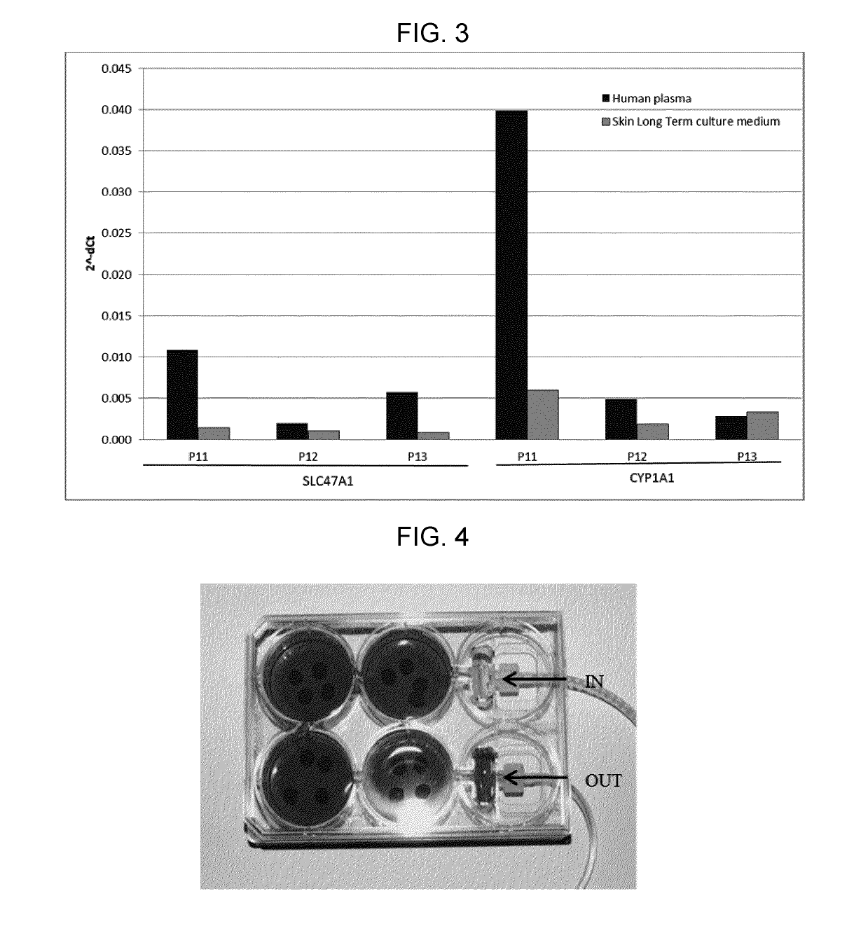

The results presented in FIG. 3 show that gene expression of SLC47A1 was higher in skin biopsies cultured in human plasma than in skin biopsies cultured in synthetic culture medium. The same result was confirmed for the three donors used.

Similarly, the results show that gene expression of CYP1A1 was higher in skin biopsies cultured in human plasma than in skin biopsies cultured in synthetic culture medium. The same result was confirmed for two out of three donors used.

Taken together, the results show that human plasma increases expression of SLC47A1 and CYP1A1 genes involved in drug transport and metabolism, indicating that the use of human plasma instead of a synthetic culture medium improves the functionality of skin organ culture model.

### Example 2

### Effect of Culture Medium Perfusion on Gene Expression in Skin Organ Culture Model.

Skin punch biopsies each 6 mm in diameter were cultured in the wells of a 6-well culture plate in a synthetic culture medium (Skin long term culture medium, Biopredic, France). In this immersed organ culture model, 4 skin biopsies were cultured per well. Each well was filled with 5 mL of culture medium. The 6-well plate was maintained in a cell incubator set at 37° C., 5% CO₂, and high humidity. Fresh culture medium was provided every day for a duration of 3 days.

In parallel, skin punch biopsies each 6 mm in diameter obtained from the same donor were cultured in a Reinnervate perfusion plate with 4 skin biopsies per well (FIG. 4).

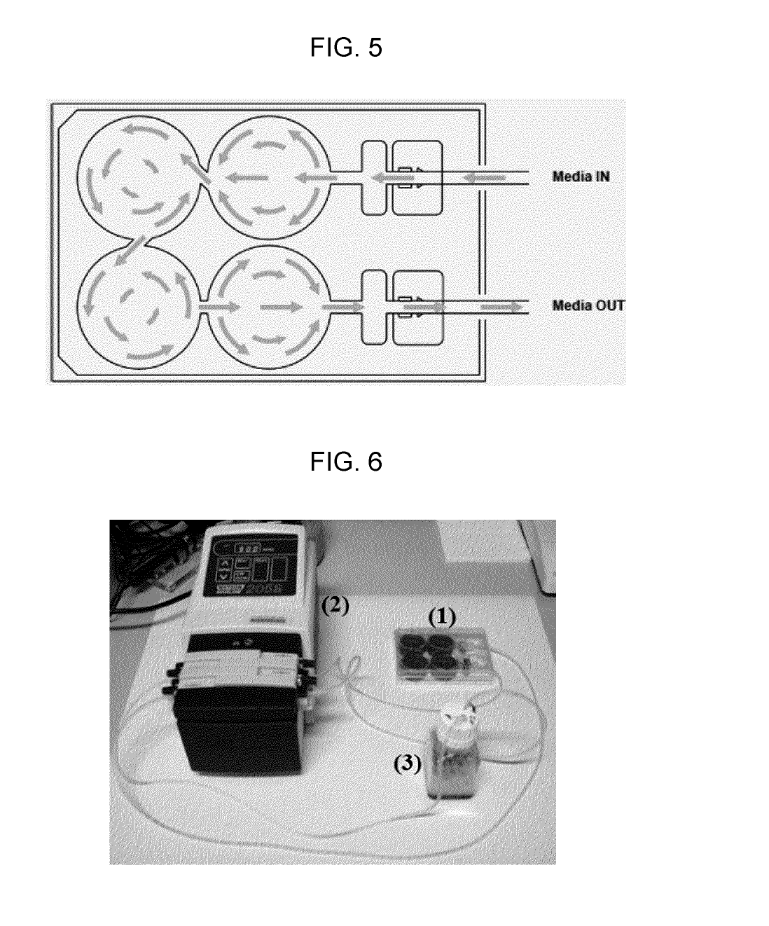

This perfusion plate allows for a unidirectional dynamic media flow and perfusion across skin biopsies, as shown in FIG. 5. Perfusion of culture medium (130 mL) was made continually for a duration of 3 days using a peristaltic pump. The same culture media, pumped from the reservoir, was continually circulated and returned through the plate repeatedly. The system (FIG. 6), which included a perfusion plate (1), a peristaltic pump (2), and a culture medium reservoir (3), was maintained in a cell incubator set at 37° C., 5% CO₂, and high humidity during the 3-day period of the experiment. Three different flow rates of the peristaltic pump were assayed: 0.1 mL/min, 6 mL/min, and 11 mL/min. Each flow rate was tested in one separate experiment using a different skin donor.

At the end of the 3-day culture period, skin biopsies were washed using sterile PBS buffer, and total RNA was extracted from the skin samples. Gene expression of some solute carrier (SLC) transporter genes (SLC47A1 and SLC47A2 coding for MATE1 and MATE2 transporters, respectively), some ATP-binding cassette (ABC) transporter genes (ABCB1, ABCC1, ABCC2, and ABCG2), and CYP1A1 and CYP1A2 was analyzed by real time PCR as described in Example 7 below.

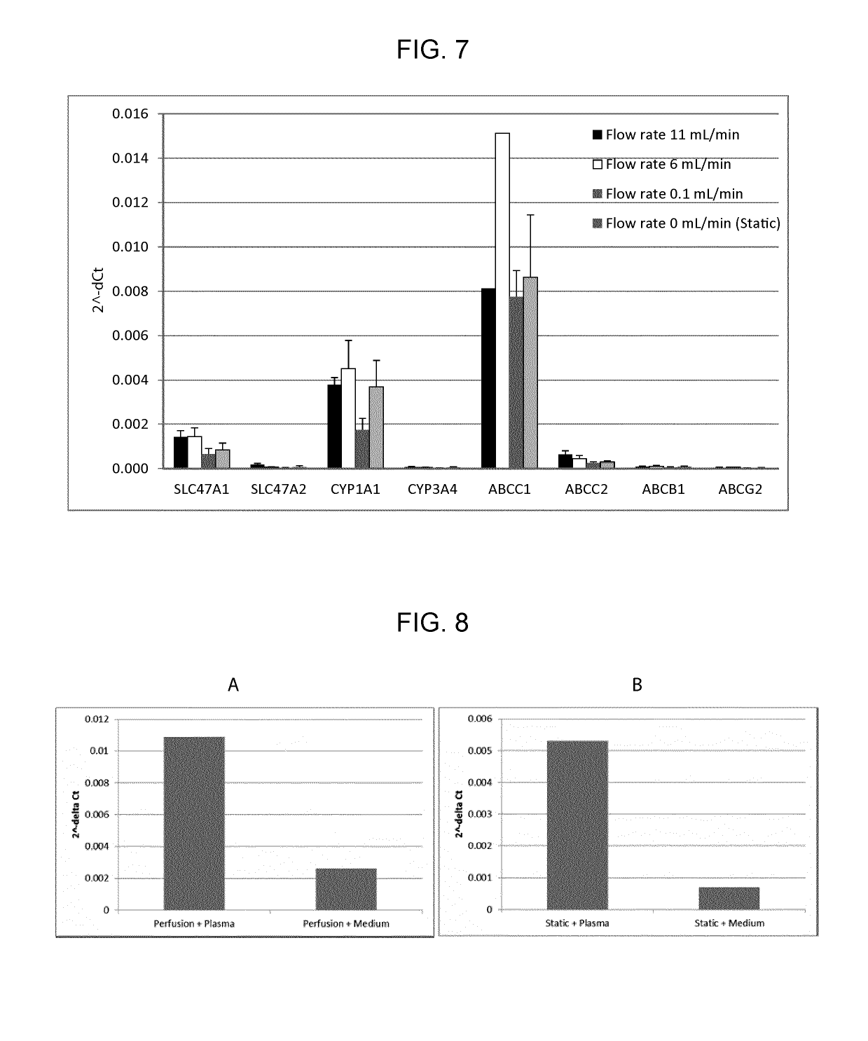

The results of the experiment presented in FIG. 7 show that the expression of most genes studied was higher in skin biopsies cultured in perfusion plate with a flow rate greater than 0.1 mL/min compared to skin biopsies cultured without perfusion (6-well plate) or with a very low flow rate of 0.1 mL/min.

Taken together, the results show that dynamic perfusion of culture medium increases the expression of the genes involved in drug transport and metabolism in human skin organ culture, indicating that the use of dynamically perfused culture medium instead of static culture enhances the conditions of human skin organ culture model.

### Example 3

### Effect of Combination of Both Culture Medium Perfusion and the Use of Human Plasma as Culture Medium on Gene Expression in Skin Organ Culture Model.

In one experiment, skin punch biopsies each 6 mm in diameter were cultured in the wells of a 6-well culture plate in a synthetic culture medium (Skin long term culture medium, Biopredic, France). In this immersed organ culture model, 4 skin biopsies were cultured per well. Each well was filled with 5 mL culture medium. The 6-well plate was maintained in a cell incubator set at 37° C., 5% CO₂, and high humidity. Fresh culture medium was provided every day for 3 days. In parallel, the same experiment was done using human plasma as culture medium instead of skin long term culture medium. Human plasma was changed every day for 3 days.

In another experiment, skin punch biopsies each 6 mm in diameter obtained from the same donor used in the experiment described above were cultured in Reinnervate perfusion plate with 4 skin biopsies per well. Perfusion of culture medium (130 mL of skin long term culture medium) was made continually for 3 days using a peristaltic pump with a flow rate of 11 mL/min. The same culture media pumped from the reservoir was continually circulated and returned through the plate repeatedly. The system including perfusion plate (1), peristaltic pump (2) as well as culture medium reservoir (3) (see FIG. 6) was maintained in a cell incubator set at 37° C., 5% CO₂, and high humidity during the 3-day period of experiment. In parallel, the same experiment was done using human plasma as culture medium instead of skin long term culture medium.

At the end of the 3-day culture period, skin biopsies were washed using sterile PBS buffer, and total RNA was extracted from skin samples. Gene expression of SLC47A1 was analyzed by real time PCR as described in Example 7 below.

### Example 4

Human Plasma with Static/Dynamic Perfusion Conditions.

The results presented in FIG. 8 obtained on one donor (P15) show that the expression of SLC47A1 gene was higher in human skin biopsies cultured in human plasma compared to human skin biopsies cultured in skin long term culture medium regardless of whether the culture conditions were dynamic (FIG. 8A) or static (FIG. 8B). This confirms the previous result (FIG. 3) showing that the use of human plasma as a culture medium increases gene expression of certain genes in human skin organ culture.

### Example 5

Dynamic Perfusion Conditions with Culture Medium/Human Plasma.

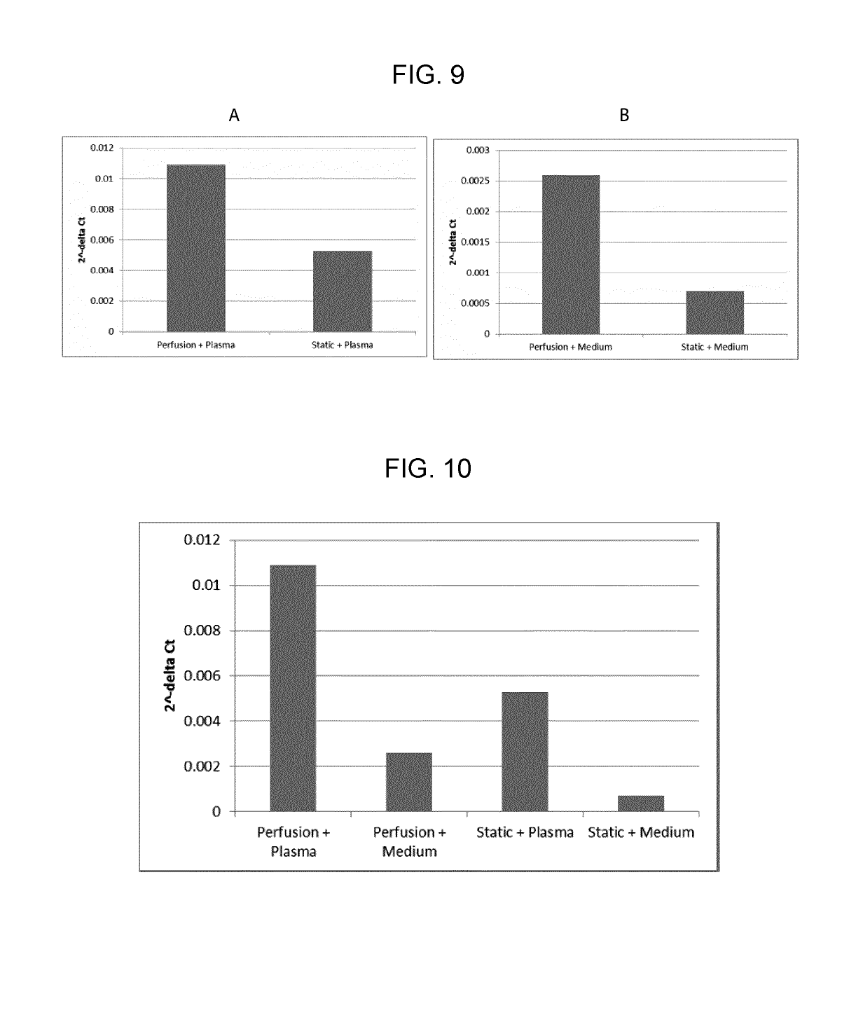

On the other hand, the results presented in FIG. 9 obtained on one donor (P15) show that the expression of SLC47A1 gene was higher in human skin biopsies cultured in dynamic perfusion conditions compared to human skin biopsies cultured under static conditions regardless of the culture medium used, i.e., human plasma (FIG. 9A) or skin long term culture medium (FIG. 9B). This confirms the previous result (FIG. 7) showing that the dynamic perfusion of culture medium increases the expression of the genes involved in drug transport and metabolism in human skin organ culture.

### Example 6

Combination of Dynamic Perfusion Conditions with Human Plasma

In addition, the results presented in FIG. 10 show that the combination of both conditions—substitution of culture medium by human plasma and use of dynamic perfusion conditions instead of static conditions—led to the highest gene expression level of SLC47A1 in human skin organ culture.

All the results obtained on human donor P15 were confirmed on another human donor P14 (data not shown).

Taken together, the results of the experiments of Examples 1-6 show that the use of human plasma associated with dynamic perfusion conditions increases the expression of genes involved in drug metabolism and transport in human skin organ culture model. The results suggest that this quick and simple system can be used to study pharmacology, dermal toxicity, and penetration and metabolism of xenobiotics in skin biopsies maintained in culture conditions very close to their natural microenvironment.

### Example 7

### RNA Extraction and Gene Expression Analysis by Real Time PCR

After homogenization of skin samples or hepatocytes in lysis buffer (Promega), total RNA was isolated using SV Total RNA Isolation System (Promega) in accordance with the instructions provided by the supplier. RNA concentrations were quantified spectrophotometrically. Quantification of mRNA expression of human SLC transporters was performed using TaqMan PCR techniques (Applied Biosystems). Experiments were carried out on a 7500 real time PCR System (Applied Biosystems) using Assay-on-Demand gene expression products. For this, 500 ng of total RNA were reverse-transcribed using the High Capacity RNA to cDNA Master Mix kit (Applied Biosystems).

PCR amplifications were performed in a total volume of 25 μL using the TaqMan Universal Master Mix (Applied Biosystems). Denaturation was performed at 95° C. for 10 min, followed by 40 PCR cycles with the following specifications: 95° C. for 15 s and 60° C. for 60 s. Glyceraldehyde-3-phosphate dehydrogenase (GAPDH) was used as a reference gene for normalization in each sample. TaqMan Gene Expression Assays from Applied Biosystems were used in the expression profiling experiments. All RT-PCR measurements were performed in triplicate. Quantification of the gene expression level of each transcript in each sample was calculated using the comparative threshold cycle (Ct) method, also called delta Ct method [3]. Briefly, expression values for target genes were normalized to the concentration of GAPDH, which showed the least variation among reference genes in our biological systems. The Ct data for target gene and GAPDH in each sample were used to create delta Ct values (Ct target gene—Ct GAPDH) giving the delta Ct. The results were expressed as 2-delta Ct.

### REFERENCES

1. Tammi R, & Maibach H. Skin Organ Culture: Why? 1987, International Journal of Dermatology, 26: 150-160
2. Companjen A R 1, van der Wel L I, Wei L, Laman J D, Prens E P. A modified ex vivo skin organ culture system for functional studies. 2001, Archives of Dermatological Research, 293:184-190
3. Atac B, Wagner I, Horland R, Lauster R, Marx U, Tonevitsky A G, Azar R P, Lindner G. Skin and hair on-a-chip: in vitro skin models versus ex vivo tissue maintenance with dynamic perfusion. 2013, Lab Chip, 13 : 3555-61
4. Livak KJ, Schmittgen TD. Analysis of relative gene expression data using real-time quantitative PCR and the 2(-Delta Delta C(T)) method. 2001, Methods, 25: 402-8

1. A skin organ culture model system comprising:
a perfusion plate (1) comprising at least one skin biopsy;
a peristaltic pump (2); and
a culture medium reservoir (3) comprising a culture medium, and connected to the perfusion plate (1) and the peristaltic pump (2),
wherein the culture medium is plasma, and the peristaltic pump (2) pumps the culture medium from the culture reservoir (3) into and out of the perfusion plate (1) at a flow rate ranging from 0.1 mL/min to 20 mL/min.

2. The skin organ culture model system according to claim 1, wherein the plasma is human plasma.

3. The skin organ culture model system according to claim 1, wherein the at least one skin biopsy is a human skin biopsy.

4. The skin organ culture model system according to claim 1, wherein the flow rate of the peristaltic pump ranges from 9 mL/min to 13 mL/min.

5. The skin organ culture model system according to claim 1, wherein the at least one skin biopsy is subjected to dynamic perfusion conditions.

6. The skin organ culture model system according to claim 1, wherein the peristaltic pump (2) pumps the culture medium from the culture reservoir (3) through the perfusion plate (1) according to a unidirectional dynamic flow.

7-9. (canceled)

10. A method of evaluating at least one effect selected from the group consisting of skin penetration (absorption and distribution) of xenobiotics; topical drug metabolism; drug-drug interactions; drug-sunscreen interactions; inflammatory skin reaction; efficacy of sunscreen or effects of UV exposure; effect of various photo-protective drugs intended to prevent or treat skin cancer; immunological function of skin cells by measuring cytokine secretion in the culture medium; efficacy and safety of cosmetic ingredients and drugs; and efficacy of cosmetic “anti-aging” skin care by measuring collagen synthesis, the method comprising maintaining the skin organ culture model system according to claim 1 in a cell incubator set at 37° C. and 5% CO₂ for a duration of 3 to 7 days.

11. A method of identifying biomarkers responsible for repair and regeneration of damaged skin, the method comprising maintaining the skin organ culture model system according to claim 1 in a cell incubator system set at 37° C. and 5% CO₂ for a duration of 3 to 7 days, and measuring a level of gene expression of one or more genes selected from the group consisting of drug transporter genes and metabolism genes.
